(19) United States
(12) Patent Application Publication
(10) Pub. No.: US 2013/0095194 A1
(43) Pub. Date: Apr. 18, 2013

(54) USE OF INHALED NITROUS OXIDE OR XENON FOR PREVENTING NEUROPATHIC PAIN CAUSED BY CANCER CHEMOTHERAPY

(71) Applicant: L'Air Liquide, Société Anonyme pour I'Etude et, Paris (FR)

(72) Inventors: Baptiste BESSIERE, Issy les Moulineaux (FR); Jan Pype, Herne (BE); Guy Simonnet, Bordeaux (FR)

(73) Assignee: L'Air Liquide, Société Anonyme pour I'Etude et I'Exploitation des Procédés Georges Claude, Paris (FR)

(21) Appl. No.: 13/647,495

(22) Filed: Oct. 9, 2012

(30) Foreign Application Priority Data
Oct. 14, 2011 (FR) 1159296

(51) Int. Cl. A61K 33/00; A61P 35/00; A61P 25/04

(52) U.S. Cl. 424/718; 424/600

(57) ABSTRACT

The invention relates to a gaseous inhalable medicament containing xenon or N₂O as active ingredient for use by inhalation for preventing and/or for treating neuropathic pain or pains caused by at least one cancer chemotherapy substance administered to a patient suffering from cancer, in particular a patient suffering from breast cancer, lung cancer, ovarian cancer, prostate cancer, colon cancer, rectal cancer or a gastric cancer or cancer of the upper aerodigestive tracts. The cancer chemotherapy substance contains one or more compounds chosen from platinum salts, taxanes, alkaloids, thalidomide and bortezomib, in particular paclitaxel, docetaxel or oxaliplatin. The effective volume proportion of nitrous oxide or of xenon is between 5 and 70%.

### CROSS REFERENCE TO RELATED APPLICATIONS

This application claims the benefit of priority under 35 U.S.C. §119 (a) and (b) to French Application No. 1159296, filed Oct. 14, 2011, the entire contents of which are incorporated herein by reference.

### BACKGROUND

The invention relates to a xenon-based or N₂O-based gaseous inhalable composition or medicament for preventing, i.e. stopping the occurrence of, or treating, i.e. completely or partially curing, or for at least minimizing, neuropathic pain caused by a cancer chemotherapy substance administered to a patient suffering from cancer.

The development of chemotherapies for treating cancers has led to a notable improvement in patient survival and often to a return to normal or virtually normal life.

However, cancer chemotherapy is generally neurotoxic and often causes considerable complications.

More specifically, the neurotoxic side effects affect the peripheral nervous system by targeting the cell body of neurons, the axon, the myelin sheath and/or the glial cells. This usually results in peripheral neuropathy caused by the chemotherapy, as recalled by C. Sioka et al., Central and peripheral nervous system toxicity of common chemotherapeutic agents; Cancer Chemother. Pharmacol.; 2009; 63; p. 761-767).

More generally, the prevalence of chemo-induced sensory neuropathies varies from 10% to 80% depending on the medicament.

Cancer chemotherapeutic agents or cancer chemotherapy substances which cause this type of neurotoxicity are in particular platinum salts, such as cisplatin, oxaliplatin and carboplatin; taxanes, such as paclitaxel and docetaxel; and alkaloids, in particular vincristine, thalidomide and bortezomib.

Neuropathic pain is consequently an important side effect for many patients, which can occur at the beginning of treatment, but also as a late consequence of the treatment, and then result in chronic pain.

Thus, the document S. Couraud et al.; Hypersensibilité aux sels de platine [Hypersensitivity to platinum salts]; Revue de Pneumo. Clinique; Vol. 64, No. 1; p. 20-26; February 2008, recalls that hypersensitivity reactions to platinum salts are frequent and sometimes serious. These effects can occur after 4 to 8 cycles of treatment. Moreover, a long interval between two courses of treatment increases the risk of reaction. The medical treatment should then consist, in simple cases, of careful reintroduction of the active ingredient, i.e. of the platinum salt used, according to a desensitization protocol, or, in severe cases, of careful replacement of the salts used with another salt, when this is possible.

In any event, it is understood that these hypersensitivity reactions to platinum salts are a real problem and must not be taken lightly.

Sometimes, neuropathy can develop even after a single drug application, in particular in the case of a treatment with oxaliplatin, as shown by M. Stengel et al.; Oxaliplatin-induced painful neuropathy—Flicker of hope or hopeless pain?; Pain 2009; 144; p. 225-226).

Similarly, taxanes are known to produce unwanted side effects, in particular neuropathies which are reflected by feelings of numbness or tingling, mainly in the hands and feet in patients.

However, up until now, no medicament or product really effective for preventing and/or curing neuropathic pain caused by cancer chemotherapy treatment has been proposed.

Moreover, document FR-A-2944969 proposes using nitrous oxide at a content between 5 and 45% by volume for treating chronic pain in humans, in particular that of dysfunctional, inflammatory, neuropathic or iatrogenic origin, in particular neuropathic pain chosen from post-shingles pain, diabetic pain, central pain, post-stroke pain, multiple sclerosis-related pain, pain related to spinal cord injury, aids-related or cancer-related pain, chemotherapy-related pain or pain of post-operative origin.

However, this document is completely silent with regard to pain resulting from treatment with one or more platinum salts, taxanes or another analogous substance of the alkaloid, thalidomide or bortezomib type.

The problem which arises is consequently that of being able to provide an effective medicament capable of preventing and/or curing, completely or partially (i.e. attenuating), a neuropathic pain (or pains) caused by a cancer chemotherapy treatment to which a patient, typically a human patient, is subjected, in particular a treatment by administration of a substance containing one or more platinum salts, such as cisplatin, oxaliplatin and carboplatin, more particularly oxaliplatin, or taxanes, alkaloids, thalidomide or bortezomib.

### SUMMARY

The solution according to the invention relates to a gaseous inhalable medicament containing xenon (Xe) or nitrous oxide (N₂O) as active ingredient for use by inhalation for preventing and/or for treating at least one neuropathic pain caused by at least one cancer chemotherapy substance administered to a patient suffering from cancer, said cancer chemotherapy substance containing one or more compounds chosen from platinum salts, taxanes, alkaloids, thalidomide and bortezomib.

In the context of the invention, it is specified that:
the term “preventing” is intended to mean stopping the occurrence of;
the term “treating” is intended to mean completely or partially curing, including decreasing, minimizing or reducing;
the term “patient” is intended to mean an individual suffering from cancer;
the term “inhalable” is intended to mean can be administered by inhalation; and
the term “%” is intended to mean a volume percentage, i.e. % by volume, unless otherwise indicated.

As appropriate, the inhalable medicament of the invention, i.e. the gaseous medicinal composition of the invention, may comprise one or more of the following technical characteristics:
the cancer chemotherapy substance contains a compound chosen from platinum salts, taxanes, alkaloids, thalidomide and bortezomib;
the cancer chemotherapy substance contains several compounds chosen from platinum salts, taxanes, alkaloids, thalidomide and bortezomib;
the cancer chemotherapy substance is chosen from platinum salts, such as cisplatin, oxaliplatin or carboplatin; taxanes, in particular paclitaxel and docetaxel; alkaloids, in particular vincristine; thalidomide; and bortezomib;
the cancer chemotherapy substance contains one or more platinum salts chosen from cisplatin, oxaliplatin and carboplatin;
the cancer chemotherapy substance contains oxaliplatin, which is commonly used in the treatment of colon or rectal cancers, in particular such as stage III colon cancer (Dukes' C stage) and the treatment of metastatic colorectal cancers;
the patient is suffering from colon or rectal cancer;
the cancer chemotherapy substance comprises at least one taxane chosen from paclitaxel (Taxol™) and docetaxel (Taxotère™). The taxanes are a class of chemotherapeutic agents which act by inhibiting the function of microtubules which are essential for cell division. Taxanes therefore block normal division of cancer cells;
the patient is suffering from breast cancer, lung cancer (non small cell), ovarian cancer or prostate cancer or from gastric cancer or cancer of the upper aerodigestive tracts;
the effective volume proportion of nitrous oxide or of xenon is between 5 and 70%, preferably less than 60%;
the effective volume proportion of nitrous oxide or of xenon is less than or equal to 50%, or even less than 40%;
it contains an effective volume proportion of nitrous oxide or of xenon of between 10 and 50%;
it contains an effective volume proportion of nitrous oxide or of xenon of between 10 and 30%;
it also contains an oxygen (O₂) volume proportion of at least 21%, typically between 21% and 50%;
it is administered by inhalation for at least 15 minutes per day, preferably at least 30 min/day, more preferably at least 1 h/day;
it is administered by inhalation for a period of time sufficient to obtain an observable effect within a day or days following its administration by inhalation;
it is administered by inhalation continuously or in fractions, i.e. several times or in several goes;
it can be coadministered with the cancer chemotherapy substance, namely in particular a cancer chemotherapy substance containing one or more compounds chosen from platinum salts, taxanes, alkaloids, thalidomide and bortezomib;
it is administered by inhalation for at least the entire period of time during which the patient is treated with the cancer chemotherapy substance. Thus, if the patient must take the cancer chemotherapy substance for 6 months for example, then the medicament is also administered for this same period of 6 months, or even a little longer in order to palliate any possible delayed effects;
it also contains an additional gas chosen from argon, helium, neon, krypton, NO, CO, hydrogen sulphide (H₂S) and nitrogen, including air;
it is coadministered with the cancer chemotherapy substance, i.e. before, during and/or after administration of the cancer chemotherapy substance;
the patient is a human being, i.e. a man or a woman, including children, adolescents or any other group of individuals, for example newborn babies or elderly individuals;
it is ready to use;
it is packaged in a gas cylinder.

In other words, according to the present invention, it is proposed to inhale nitrous oxide or xenon in order to prevent or treat in a long-lasting manner, i.e. for one or more days, weeks or months, the neuropathic pain that may be caused by one (or more) cancer chemotherapy substance, i.e. an anticancer agent, chosen from platinum salts, taxanes, alkaloids, thalidomide and bortezomib, in particular caused by a substance such as oxaliplatin.

The invention is therefore based on coadministration of the cancer chemotherapy substance chosen from platinum salts, taxanes, alkaloids, thalidomide and bortezomib, and of the gas mixture comprising nitrous oxide or xenon in order to prevent or treat chemo-induced neuropathic pain.

The concentration and/or the duration of administration most suitable for a given patient can be chosen empirically by the care staff, for example depending on the patient's state of health or physical condition, on the severity of the pain, on the patient's sex and age, on the type of cancer, on the type of anticancer agent coadministered, etc.

The gas mixture or medicament of the invention may be used in the context of a therapeutic treatment method, in which the gas mixture is administered by inhalation, for example by means of a breathing mask, either directly connected to a source of N₂O or of xenon at the required concentration, for example a ready-to-use gas cylinder, or else to the outlet of a gas mixer supplied by several sources of gas (O₂, N₂O or Xe, etc.) so as to obtain the desired mixture; or connected to a respiratory ventilator supplied with the desired gas(es). In other words, the present invention is therefore based on the use of a therapeutic gas mixture containing N₂O or xenon as active ingredient in an effective proportion, for producing an inhalable medicament with curative or preventive effects, intended for treating neuropathic pain caused by a substance used in the context of cancer chemotherapy and administered to a patient suffering from cancer, said substance being chosen from platinum salts, taxanes, alkaloids, thalidomide and bortezomib, in particular a substance of the oxaliplatin type.

The nitrous oxide (N₂O) or the xenon are therefore, according to the invention, used in the context of a therapeutic treatment method in which the N₂O or the xenon is administered to a mammal, in particular a human patient, by inhalation, for preventing or treating neuropathic pain caused by an anticancer substance used as cancer chemotherapy administered to said patient.

### BRIEF DESCRIPTION OF THE DRAWINGS

The present invention will now be understood more fully by means of the following description and the following examples, which are given purely by way of illustration and with reference to the appended figures, among which:

FIG. 1 represents a diagram of the oxaliplatin molecule; and

FIG. 2 represents the effects of N₂O and of xenon on neuropathic pain caused by oxaliplatin in rats.

### DESCRIPTION OF PREFERRED EMBODIMENTS

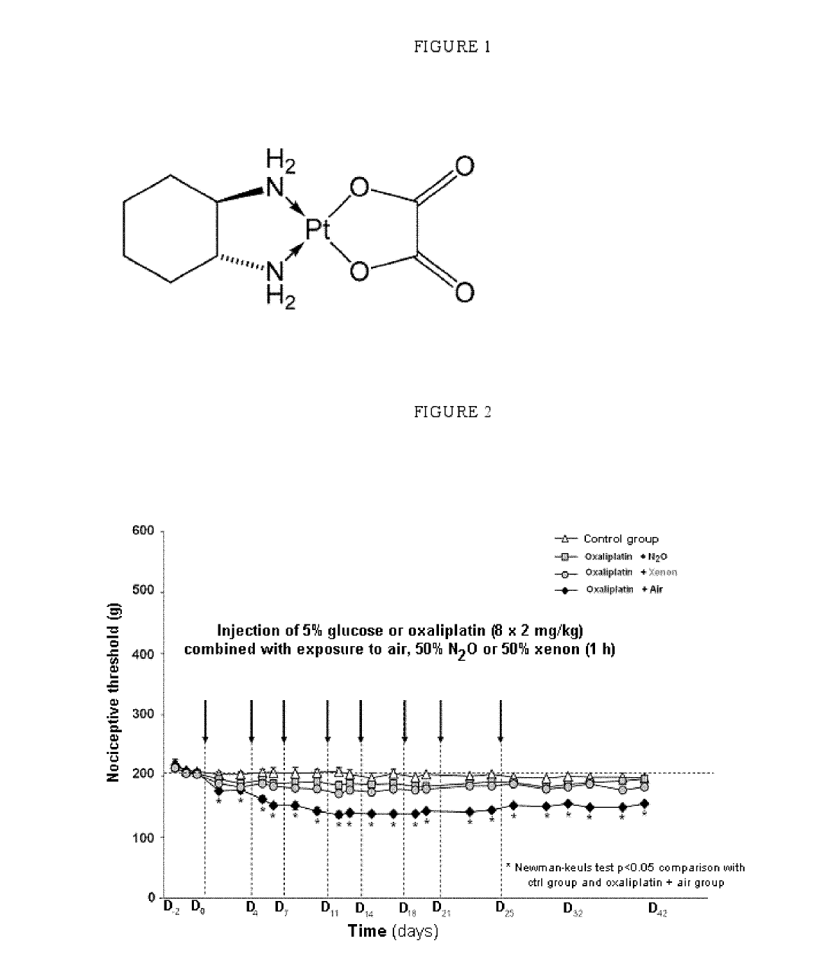

FIG. 1 represents a diagram of the oxaliplatin molecule that was used in the trials hereinafter as anticancer substance.

Oxaliplatin is commonly used for combating cancers, in particular cancers of the rectum and colon, since it allows inhibition of DNA synthesis and replication by formation of intra-strand bridges between adjacent guanines, which is a treatment of choice in combating cancers of this type. This molecule unfortunately has the drawback of inducing neuropathic pain in the cancer patients to whom it is administered.

As a result, in order to demonstrate the efficacy of the N₂O-based or xenon-based gas according to the invention in combating this type of neuropathic pain caused by oxaliplatin, comparative trials were carried out under the following conditions.

A preclinical study in rats was carried out.

In order to mimic the clinical use as closely as possible, a series of 8 intraperitoneal injections of oxaliplatin were given every 3 to 4 days, as illustrated in FIG. 2. This model is well known in the literature and gives an account of chemo-induced neuropathic pain.

The pain thresholds were evaluated by means of a Randall-Selitto device by applying an increasing pressure to the paw of the rat until the animal lets out a cry. The corresponding weight in grams is then noted (the Y-axis in FIG. 2). Thus, the earlier the animal cries out, for a low weight, the more pain it has.

In order to evaluate the effects of 50% nitrous oxide and of 50% xenon (% by volume) on the neuropathic pain caused by oxaliplatin, 4 groups of animals, in a proportion of 8 rats per group, were used (see FIG. 2):
group A or control group (white triangle): the animals received a controlled solution of 5% glucose in place of the oxaliplatin and the administrations were combined with an N₂/O₂ (50%-50%) control gas mixture for 1 hour;

FIG. 2 shows exemplary results of preclinical animal model testing wherein:
group B or Oxaliplatin+Air group (black diamond): the animals received oxaliplatin (2 mg/kg) and the administrations were combined with an N₂/O₂ (50%-50%) control gas mixture for 1 hour;
group C or Oxaliplatin+N₂O group (grey square): the animals received a solution containing oxaliplatin (2 mg/kg) and the administrations were combined with a gas mixture containing N₂O/O₂ (50%-50%) for 1 hour; and
group D or Oxaliplatin+Xenon group (grey circle): the animals received a solution containing oxaliplatin (2 mg/kg) and the administrations were combined with a gas mixture containing Xe/O₂ (50%-50%) for 1 hour.

### EXAMPLES

FIG. 2, groups A and B are given by way of comparison, while groups C and D are according to the present invention.

Following the administrations of oxaliplatin, the pain thresholds of group B (oxaliplatin+air) are significantly decreased, namely approximately 150 g compared with 200 g in group A, revealing hypersensitivity to pain or hyperalgesia in the rats of group B which did receive oxaliplatin. This confirms that this molecule has the drawback of inducing neuropathic pain.

The significance of the results is represented by the star close to the black diamonds in FIG. 2.

Interestingly, it is noted that this hypersensitivity to pain persists well beyond the final administration of oxaliplatin. Indeed, as can be seen in FIG. 2, the final administration took place on D25 and the hypersensitivities to pain are still present more than 2 weeks later (D42).

Conversely, groups C and D in which the rats inhaled N₂O or xenon after administration of oxaliplatin exhibit no sign of hypersensitivity to pain.

Thus, the respective curves (grey square and grey circle) for groups C and D are not different from the curve of group A (white triangle) acting as a control group.

In other words, the administrations of gas mixture containing N₂O or xenon made it possible, surprisingly, to prevent or treat the hypersensitivities to pain caused by the successive administrations of oxaliplatin.

This constitutes a notable advance from the medical point of view since, applied to human patients, these N₂O or xenon inhalations would make it possible, finally, to relieve the pain experienced by cancer patients undergoing chemotherapies based on oxaliplatin or on other anticancer substances.

Moreover, even more surprisingly, the effect of the xenon or of the N₂O lasts over time since, even following the final administration of oxaliplatin, the long-term hypersensitivity to pain is completely prevented. In fact, these trials show that the preventive effect of N₂O or of xenon lasts more than 2 weeks.

These trials clearly demonstrate that xenon and N₂O administered by inhalation make it possible to prevent or treat neuropathic pain caused by cancer chemotherapy substances, such as oxaliplatin.

Xenon and N₂O gases mixed with oxygen or with air, for example, can consequently be used for treating, preventing, decreasing, minimizing, etc., neuropathic pain which is a negative side effect of the use of certain anticancer substances, such as platinum salts, taxanes, alkaloids, thalidomide and bortezomib, which can be administered alone or in the form of a “cocktail” of several substances.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A method of treating a neuropathic pain in a cancer patient undergoing chemotherapy, the method comprising the step of administering a gaseous inhalable medicament containing xenon or N₂O as active ingredient by inhalation to thereby prevent and/or for treat the neuropathic pain, wherein the neuropathic pain is induced by at least one cancer chemotherapy substance administered to the patient suffering from cancer, said cancer chemotherapy substance being chosen from platinum salts, taxanes, alkaloids, thalidomide and bortezomib.

2. The method of claim 1, wherein the cancer chemotherapy substance contains several compounds chosen from platinum salts, taxanes, alkaloids, thalidomide and bortezomib.

3. The method of claim 1, wherein the cancer chemotherapy substance comprises one or more platinum salts chosen from cisplatin, oxaliplatin and carboplatin.

4. The method of claim 1, wherein the cancer chemotherapy substance comprises oxaliplatin.

5. The method of claim 1, wherein the cancer chemotherapy substance comprises at least one taxane chosen from paclitaxel and docetaxel.

6. The method of claim 1, wherein an effective volume proportion of nitrous oxide or of xenon in the gaseous inhalable medicament is between 5 and 70%.

7. The method of claim 1, wherein the gaseous inhalable medicament also contains an oxygen (O₂) volume proportion of at least 21%.

8. The method of claim 1, wherein the gaseous inhalable medicament also contains an additional gas chosen from argon, helium, neon, krypton, NO, CO, hydrogen sulphide (H₂S) and nitrogen.

9. The method of claim 1, wherein the gaseous inhalable medicament is coadministered with the cancer chemotherapy substance.

10. The method of claim 1, wherein the patient is a human being.

11. The method of claim 1, wherein the gaseous inhalable medicament contains an effective volume proportion of nitrous oxide or of xenon of between 10 and 50%.

12. The method of claim 1, wherein the patient is suffering from colon or rectal cancer.

13. The method of claim 1, wherein the patient is suffering from breast cancer, lung cancer (non small cell), ovarian cancer or prostate cancer or from a gastric cancer or a cancer of the upper aerodigestive tracts.

14. The method of claim 1, wherein the gaseous inhalable medicament is administered by inhalation for at least 15 minutes per day.
